(19) United States
(12) Patent Application Publication
(10) Pub. No.: US 2013/0211244 A1
(43) Pub. Date: Aug. 15, 2013

(54) Methods, Devices, Systems, Circuits and Associated Computer Executable Code for Detecting and Predicting the Position, Orientation and Trajectory of Surgical Tools

(71) Applicant: SURGIX LTD., (US)

(72) Inventor: Ram Nathaniel, Tel Aviv (IL)

(73) Assignee: SURGIX LTD., Herzeliya Pituach (IL)

(21) Appl. No.: 13/748,698

(22) Filed: Jan. 24, 2013

Related U.S. Application Data
(60) Provisional application No. 61/590,432, filed on Jan. 25, 2012.

(51) Int. Cl. A61B 5/00; A61B 5/06; A61B 5/055; A61B 6/00; A61B 6/03

(52) U.S. Cl. CPC A61B 5/7264; A61B 6/485; A61B 6/032; A61B 5/055; A61B 5/062; A61B 5/064; A61B 5/743; A61B 6/52; A61B 6/463
USPC 600/424

(57) ABSTRACT

The present invention includes methods, devices, systems, circuits and associated computer executable code for detecting and predicting the position and trajectory of surgical tools. According to some embodiments of the present invention, images of a surgical tool within or in proximity to a patient may be captured by a radiographic imaging system. The images may be processed by associated processing circuitry to determine and predict position, orientation and trajectory of the tool based on 3D models of the tool, geometric calculations and mathematical models describing the movement and deformation of surgical tools within a patient body.

### PRIORITY CLAIM

The present application claims priority from Provisional Patent Application No. 61/590,432, titled: “A Method Device and System for Detecting the Position and Trajectory of Surgical Tools”, filed by the inventor of the present application on Jan. 25, 2012.

### FIELD OF THE INVENTION

The present invention relates generally to the field of medical imaging. More specifically, the present invention relates to methods, devices, systems, circuits and associated computer executable code for detecting and predicting the position, orientation and trajectory of surgical tools.

### BACKGROUND

In modern surgery, a plethora of invasive tools are used regularly to facilitate a wide variety of procedures within the human body. Such tools include drills, needles, guides, lasers, blades and many more. These tools are being used, among other things, for reaching the target positioning of implants, for fixating an anatomical element during trauma surgeries, for acting as a lead for other actions such as placement of cannulated screws, etc. In many cases the tools are very thin and flexible and may bend or be otherwise distorted during an operation. When the tool bends it may take a curved path which is initially unnoticeable by the surgeon but may eventually end up at a place other than its intended target position. Clearly, during such operations, there is a need to monitor and track the position of tools within a patient's body, in real time.

The tracking of tools inside a patient's body is currently done by continuous x-ray which displays continuously on a fluoroscope or real-time digital x-ray an image of the patient's organ along with an image of the tool's location relative to the patient's organs.

One of the most popular surgical tools is the thin drill or guide wire, in all its variations and forms (sometimes referred to as the Kirschner wire). All flexible drills, guide wires and needles, in all shapes and sizes shall be referred hereinafter as “tools”.

In orthopedic surgeries, different guides are used, among other things, for reaching the target positioning of implants, for fixating an anatomy during trauma surgeries, and for acting as a lead for other actions such as placement of annulated screws.

Since the guides have some flexibility they tend to bend when the orthopedic surgeon applies force while drilling into a bone. Sometimes guides bend by accident, when a guide is deflected off a more rigid part of the bone and takes on a curved path, or when the surgeon, unintentionally changes the direction in which he holds the power drill while drilling. In other cases, the surgeon causes the drill to bend on purpose, while trying to change the path during drilling, or even bent by hand. One of the problems caused by bent guides is that surgeons have a hard time guessing the guide trajectory. Sometimes, the surgeon is unaware of the bending altogether. They find themselves surprised by the path the drilling takes and have to pull the guide out and try drilling again.

There is clearly a need for better and more accurate methods and systems for monitoring and tracking tools within a patient body.

### SUMMARY OF THE INVENTION

The present invention includes methods, devices, systems, circuits and associated computer executable code for detecting and predicting the position and trajectory of surgical tools. According to some embodiments of the present invention, there may be provided a radiographic imaging system, such as a fluoroscope or real-time digital X-Ray or CT or MRI, or, according to further embodiments, existing medical imaging systems may be functionally associated with methods, devices, systems, circuits and associated computer executable code for detecting the position and trajectory of surgical tools, according to embodiments of the present invention. According to further embodiments, methods, devices, systems, circuits and associated computer executable code for detecting the position and trajectory of surgical tools may comprise: an image processor, a system controller, an optional rendering module and/or display(s) and/or ancillary components. According to some embodiments of the present invention, the radiographic imaging system may capture images of a patient, including one or more organs and/or tissues in treatment along with the surgical tool being used on, or otherwise in proximity with, the organs or tissues.

According to some embodiments of the present invention, the image processor may be adapted to receive one or more images from the radiographic imaging system and to identify/detect the tool or certain points or markers of the tool within the image. According to further embodiments, the image processor may also be adapted to identify anatomical elements within the image. According to some embodiments of the present invention, the system controller may be adapted to receive the two dimensional appearance of the projected tool or of points or markers on the tool from the image processor, and the physical information regarding the tool, and identify and/or correlate those points on the tool and determine, calculate or estimate the tool's position and/or bending and/or orientation and/or expected trajectory. According to some embodiments of the present invention, the optional rendering module may receive position and/or bending and/or expected trajectory information relating to the tool from the system controller and render the tool's position and/or bending and/or expected trajectory, and send the image to a display to be displayed as an overlay on the tissue image.

In some embodiments of the present invention, the surgical tool may contain markers which appear within a radiographic image and may be identified by the image processor and/or controller. The surgical tool may be a tool such as a drill, a needle, a guide wire, or a blade. In some embodiments of the present invention, the markers may be made of a material visible in radiographic images or otherwise have an appearance identifiable in a radiographic image.

According to some embodiments of the present invention, the system controller, and/or image processing logic functionally associated therewith, may determine the tool's position by matching the captured image of the tool to a stored image or model (e.g. mathematical model) of the tool (skeleton) digitally stored in a repository of possible tool images or other tool related parameters. According to some embodiments of the present invention, the system controller may detect and possibly determine an extent of tool bending by identifying variation in expected spatial relationships between points on the tool. According to some embodiments of the present invention, the system controller may predict the expected trajectory of the tool by extrapolating a deflection path of the tool.

According to further embodiments, the system controller, and/or image processing logic functionally associated therewith may be further adapted to determine, or assist in determining, the position and/or orientation of a surgical tool based on mathematical models and formulas describing: (1) the movement of tools within a human anatomy, and (2) the deformation (e.g. bending) of tools within a human anatomy. According to further embodiments, such models and formulas may be tool specific and may yet further provide for anatomical data relating to the patient and/or organ/anatomical-element in contact with the tool.

According to yet further embodiments, the system controller, and/or image processing logic functionally associated therewith may be further adapted to determine, or assist in determining, the expected position and/or orientation of a surgical tool (i.e. a trajectory and/or vector of expected movement of the tool and/or its components) based on mathematical models and formulas describing: (1) the movement of tools within a human anatomy, and (2) the deformation (e.g. bending) of tools within a human anatomy. According to further embodiments, such models and formulas may be tool specific and may yet further provide for anatomical data relating to the patient and/or organ/anatomical-element expected to be in contact with the tool.

According to yet further embodiments, the system controller, and/or image processing logic functionally associated therewith may be yet further adapted to extrapolate data relating to the above described mathematical models and formulas from previous tool tracking performed by the system and current tool tracking being performed by the system.

Such models and formulas and modifications/updates/profiles for these models may be stored in a functionally associated data storage.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is an illustration of an exemplary patient leg and surgical tool being captured by an exemplary radiographic imaging device, all in accordance with some embodiments of the present invention;

FIG. 2 is an illustration of an exemplary radiographic image captured by the imaging device in FIG. 1, all in accordance with some embodiments of the present invention;

FIG. 3 is a functional block diagram of an exemplary system for detecting and predicting the position and trajectory of surgical tools, according to some embodiments of the present invention;

FIG. 4 is a simplified illustration of an appearance of an exemplary straight tool in an exemplary radiographic image, in accordance with some embodiments of the present invention;

FIG. 5 is an illustration of an expected trajectory of the exemplary tool in FIG. 4, in accordance with some embodiments of the present invention;

FIG. 6 is a simplified illustration of an appearance of an exemplary bent tool in an exemplary radiographic image, in accordance with some embodiments of the present invention;

FIG. 7 is an illustration of an expected trajectory of the exemplary tool in FIG. 6, in accordance with some embodiments of the present invention;

FIG. 8 is a simplified illustration of an exemplary tool including a marker and its appearance in an exemplary radiographic image, all in accordance with some embodiments of the present invention;

FIG. 9 is a simplified illustration of an exemplary vertically bent tool including a marker and its appearance in an exemplary radiographic image, all in accordance with some embodiments of the present invention;

FIG. 10 is a simplified illustration of an exemplary diagonally bent (horizontally and vertically) tool including a marker and its appearance in an exemplary radiographic image, all in accordance with some embodiments of the present invention;

FIGS. 11, 12,

13 and 14 are illustrations of exemplary geometric calculations and measurements being performed on exemplary radiographic images of tools, all in accordance with some embodiments of the present invention; and

FIG. 15 is an illustration of an exemplary tool including markers and the appearance of the markers in an exemplary radiographic image, all in accordance with some embodiments of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, components, circuits and algorithms have not been described in detail so as not to obscure the present invention.

Unless specifically stated otherwise, as apparent from the following discussions, it is appreciated that throughout the specification discussions utilizing terms such as “processing”, “computing”, “calculating”, “determining”, or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Embodiments of the present invention may include apparatuses for performing the operations herein. This apparatus may be specially constructed for the desired purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

The present invention includes methods, devices, systems, circuits and associated computer executable code for detecting and predicting the position and trajectory of surgical tools. According to some embodiments of the present invention, there may be provided a radiographic imaging system, such as a fluoroscope or real-time digital X-Ray or CT or MRI, or, according to further embodiments, existing medical imaging systems may be functionally associated with methods, devices, systems, circuits and associated computer executable code for detecting the position and trajectory of surgical tools, according to embodiments of the present invention. According to further embodiments, methods, devices, systems, circuits and associated computer executable code for detecting the position and trajectory of surgical tools may comprise: an image processor, a system controller, an optional rendering module and/or display(s) and/or ancillary components. According to some embodiments of the present invention, the radiographic imaging system may capture images of a patient, including one or more organs and/or tissues in treatment along with the surgical tool being used on, or otherwise in proximity with, the organs or tissues.

According to some embodiments of the present invention, the image processor may be adapted to receive one or more images from the radiographic imaging system and to identify/detect the tool or certain points or markers of the tool within the image. According to further embodiments, the image processor may also be adapted to identify anatomical elements within the image. According to some embodiments of the present invention, the system controller may be adapted to receive the two dimensional appearance of the projected tool or of points or markers on the tool from the image processor, and the physical information regarding the tool, and identify and/or correlate those points on the tool and determine, calculate or estimate the tool's position and/or bending and/or orientation and/or expected trajectory. According to some embodiments of the present invention, the optional rendering module may receive position and/or bending and/or expected trajectory information relating to the tool from the system controller and render the tool's position and/or bending and/or expected trajectory, and send the image to a display to be displayed to a user, possibly as an overlay on the tissue image.

In some embodiments of the present invention, the surgical tool may contain markers which appear within a radiographic image and may be identified by the image processor and/or controller. The surgical tool may be a tool such as a drill, a needle, a guide wire, or a blade. In some embodiments of the present invention, the markers may be made of a material visible in radiographic images or otherwise have an appearance identifiable in a radiographic image.

According to some embodiments of the present invention, the system controller, and/or image processing logic functionally associated therewith, may determine the tool's position by matching the captured image of the tool to a stored image or model of the tool (skeleton) digitally stored in a repository of possible tool images or other tool related parameters. According to some embodiments of the present invention, the system controller may detect and possibly determine an extent of tool deformation (e.g. bending) by identifying variation in expected spatial relationships between points on the tool. According to some embodiments of the present invention, the system controller may predict the expected trajectory of the tool by extrapolating a deflection path of the tool.

The present invention describes a system, device and method for tracking the location, identifying the bending, and estimating or predicting the trajectory of tools such as surgical tools near or inside a patient's body during, for instance, orthopedic surgeries.

One of the most popular surgical tools is the thin drill or guide wire, in all its variations and forms (sometimes referred to as the Kirschner wire). All flexible drills, guide wires and needles, in all shapes and sizes and other medical tools shall be referred hereinafter as “tool”.

The present invention will describe tracking surgical tools during orthopedic surgeries but the scope of the invention should not be limited and the invention may be applied to any other type of medical procedure or tool, and/or to other fields in which tracking the location, identifying any bending, and estimating or predicting the trajectory of tools may be needed.

According to some embodiments of the present invention there may be an image processing unit adapted to receive a radiographic image (as for example an x-ray image), analyze the image, and identify the tool and its location within the image. According to some other embodiments of the present invention there may be an image processing unit adapted to receive a radiographic image (as for example an x-ray image), and information about the tool's location, and identify the tool within the image. According to some embodiments of the present invention the information about the tool's location may be provided by manual input such as by a pointing device like a mouse, a touch screen or any other type of pointing device or any other type of manual input. According to some embodiments of the present invention the image processing unit may receive information about the tool's location from another system or device and/or may determine the tools location using automated object recognition.

According to some embodiments of the present invention once the image processing unit identified the tool, either automatically by analyzing the image, or by manual input, or as an input from another system or device, the image processing unit may identify certain points on the tool such as the tool's tip at the distal end and the tool's grip at the proximal end.

According to some embodiments of the present invention the tool may have markers that can be identified in the radiographic image. In some embodiments of the present invention, the markers may be made of a material visible in radiographic images or otherwise have an appearance identifiable in a radiographic image.

According to some embodiments of the present invention the image processing unit may identify in the radiographic image the markers on the tool.

According to some embodiments of the present invention there may be a system controller adapted to receive from the image processing unit information about the location of certain points on the tool. According to some embodiments of the present invention the information received by the system controller from the image processing unit may include the location of the tip of the tool and/or the location of the tool's grip and/or the location of different markers on the tool.

According to some embodiments of the present invention the system controller may receive from an external input, information about the tool such as the tool's shape, the tools' dimensions, and/or location of markers on the tool.

According to some preferred embodiments of the present invention the system controller may calculate and determine if the tool is deformed (e.g. bent). According to some embodiments of the present invention the system controller may calculate and determine the amount and direction of the tool's deformation (e.g. bending) in a three dimensional coordinate set. According to some embodiments of the present invention the bending calculation may be done by correlating the tool's image received from the image processing unit with the tool's shape received from the external input or from models of the tool stored in an associated database. According to some embodiments of the present invention the bending calculation may be done by correlating the locations of different points on the tool received from the image processing unit with the corresponding points received from the external input or in the model.

According to some embodiments of the present invention the system controller may calculate and estimate the expected trajectory of the tool. According to some embodiments of the present invention the calculation and estimation of the expected trajectory of the tool may be done by extrapolating the trajectory of the tool at or near the distal end of the tool. According to some embodiments of the present invention the system controller may use physical information about the tool (such as the tool's elasticity) for calculating the expected trajectory

According to some embodiments of the present invention the system controller may alert the surgeon that the tool is deformed (e.g. bent). According to some embodiments of the present invention the system controller may provide the surgeon information as to the level of bending. According to some embodiments of the present invention the system controller may provide the surgeon information as to the direction of the bending in a three dimensional coordinate set. According to some embodiments of the present invention the surgeon may determine certain thresholds of bending and directions above which the system controller will set an alarm. According to further embodiments, thresholds of bending and directions above which the system controller will set an alarm may be included in models of the tool used by the system.

According to some embodiments of the present invention there may be a rendering module adapted to receive the tool's shape and bending information and optionally the expected trajectory of the tool from the system controller, and render an image of the tool and optionally the expected trajectory as an overlay on the x-ray image of the organ being operated.

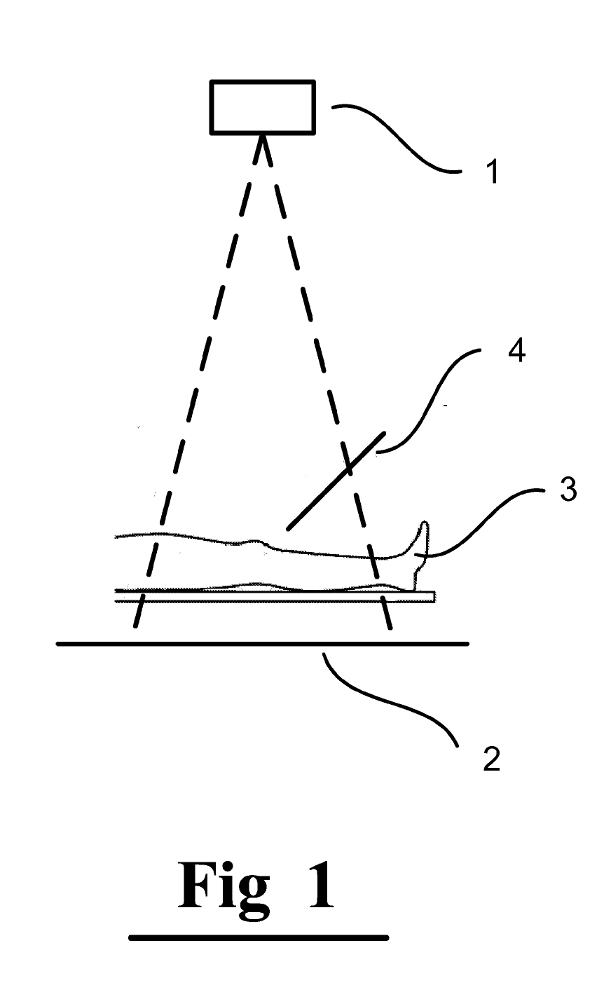

FIG. 1 is an illustration of a basic exemplary application of the system according to some embodiments of the present invention. X-ray source (1) emits electromagnetic radiation through the leg being operated (3) and the tool (4), to create an x-ray image of the leg and tool on the digital x-ray detector (2).

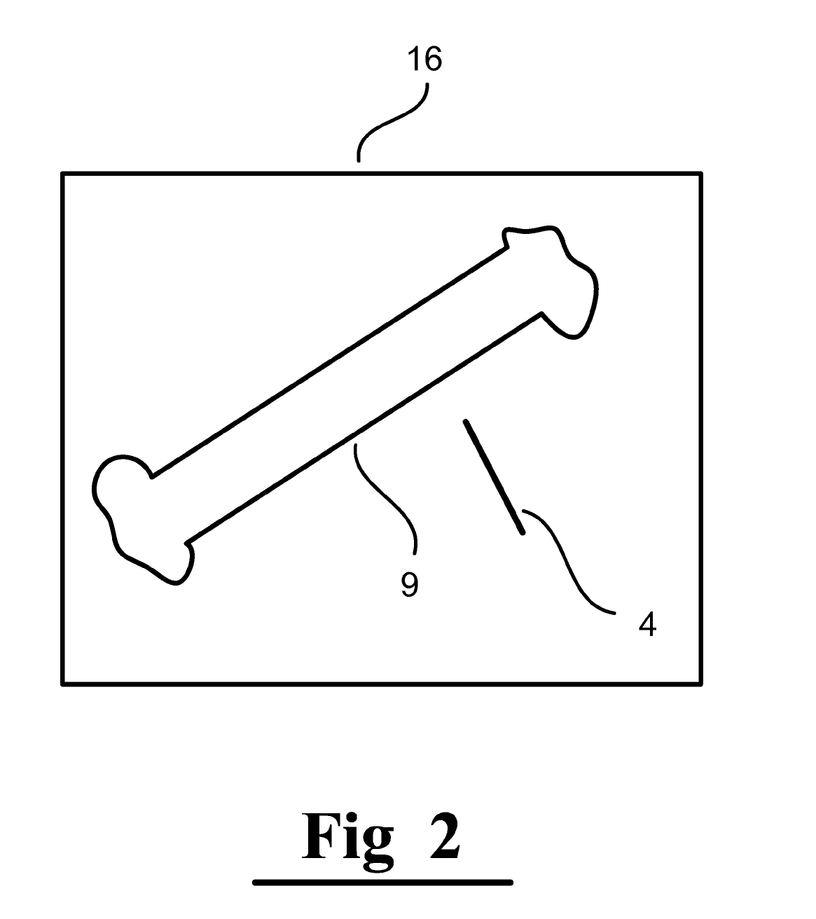

FIG. 2 is an illustration of an exemplary radiographic image captured by the imaging device in FIG. 1. In the x-ray image (16) a projection of the tool (4) can be seen along with a projection of the bone (9) onto which the tool is going to operate.

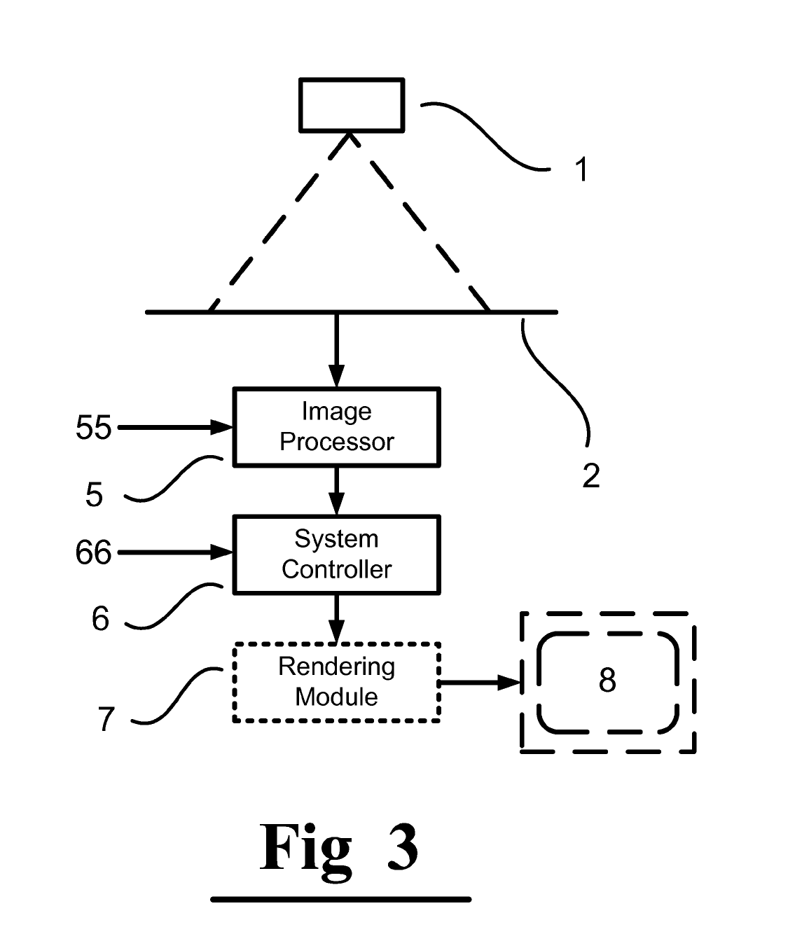

FIG. 3 is a functional block diagram of an exemplary system for detecting and predicting the position and trajectory of surgical tools, in accordance with some embodiments of the present invention. X-ray source (1) projects electromagnetic radiation onto the digital x-ray detector (2). Image processor (5) may receive the image from the x-ray detector (2) and optionally the tool's location from input 55. The tool's location can be entered manually or from another system or device and/or determined automatically. The image processor may process the image and identify the location of certain points on the tool as for example the tool's tip or markers on the tool. The system controller (6) may receive the location of points on the tool from the image processor (5) and the tool's shape and the location of points and markers on the tool through input 66.

The system controller may correlate the points received from the image processor with the corresponding points received through input 66 and may calculate the tool's position and/or orientation in a three dimensional coordinate set. The system controller may further calculate and determine if the tool is bent and may alert the surgeon of such bending. The system controller may further calculate and determine the amount of bending of the tool and the direction towards which the tool is bent. The system controller may further receive additional physical information characterizing the tool, such as the tool's flexibility, through input 66 and may further calculate and predict the expected trajectory of the tool, this may be done by extrapolating the curvature of the tool near its tip or by any other formula that may use as its input, for example, the tool's shape at rest, the current tool's bent shape, physical information characterizing the tool such as its flexibility. There may be an optional rendering module (7) that may receive the tool's location and orientation and/or bending information and/or predicted trajectory of the tool from the system controller, and render a two or three dimensional image of the tool as an overlay on top of the x-ray image of the organ being operated. The x-ray image along with the rendered image of the tool and the expected trajectory of the tool may be displayed on a monitor (8).

According to some embodiments, positional and orientational information regarding the tool may be extrapolated by comparing two dimensional projections of three dimensional models of the tool to the 2D appearances of the tool in the radiographic images.

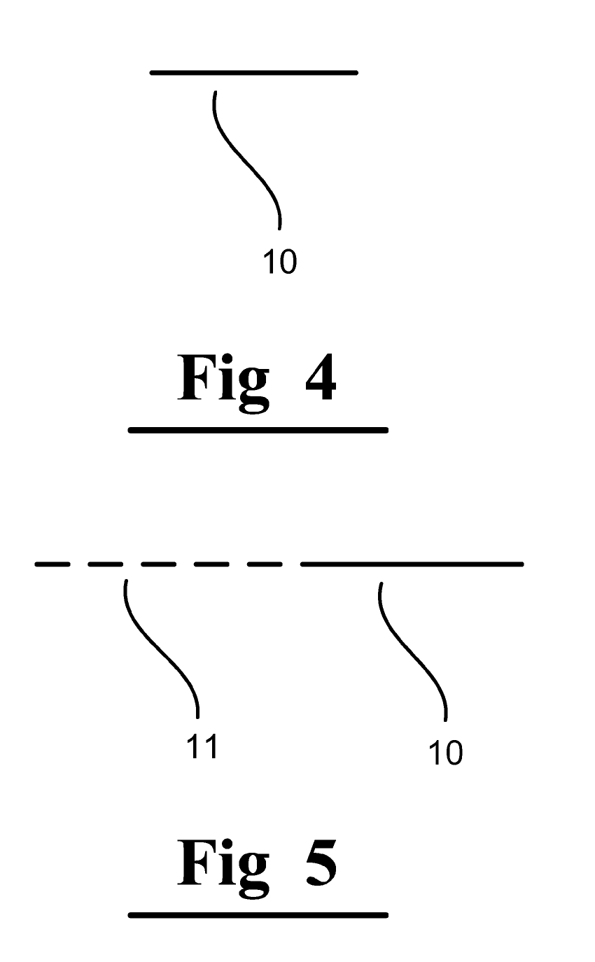

FIG. 4 is a simplified illustration of an appearance of an exemplary straight tool in an exemplary radiographic image, in accordance with some embodiments of the present invention.

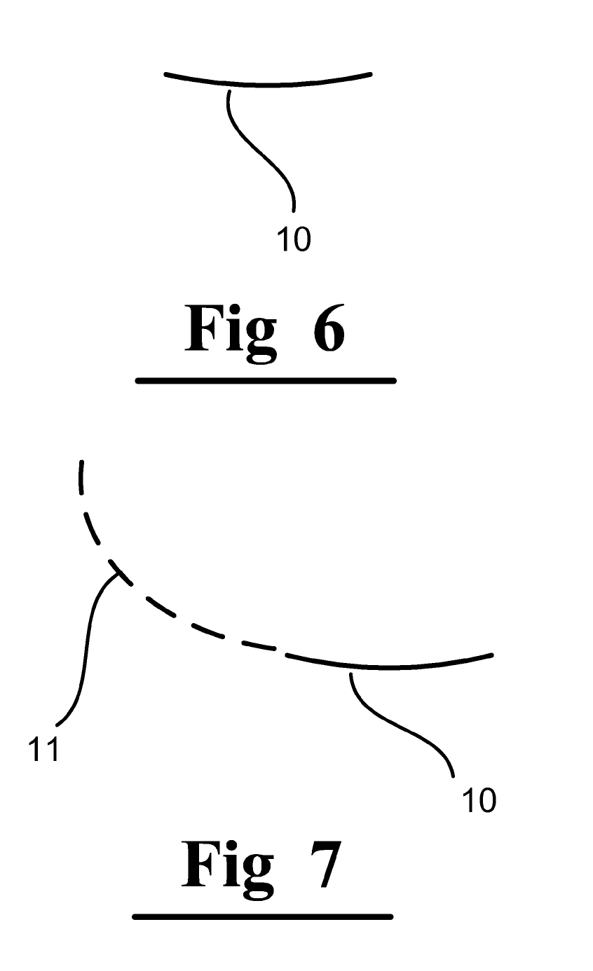

FIG. 5 is an illustration of an expected trajectory (11) of the exemplary tool (10) in FIG. 4, in accordance with some embodiments of the present invention.

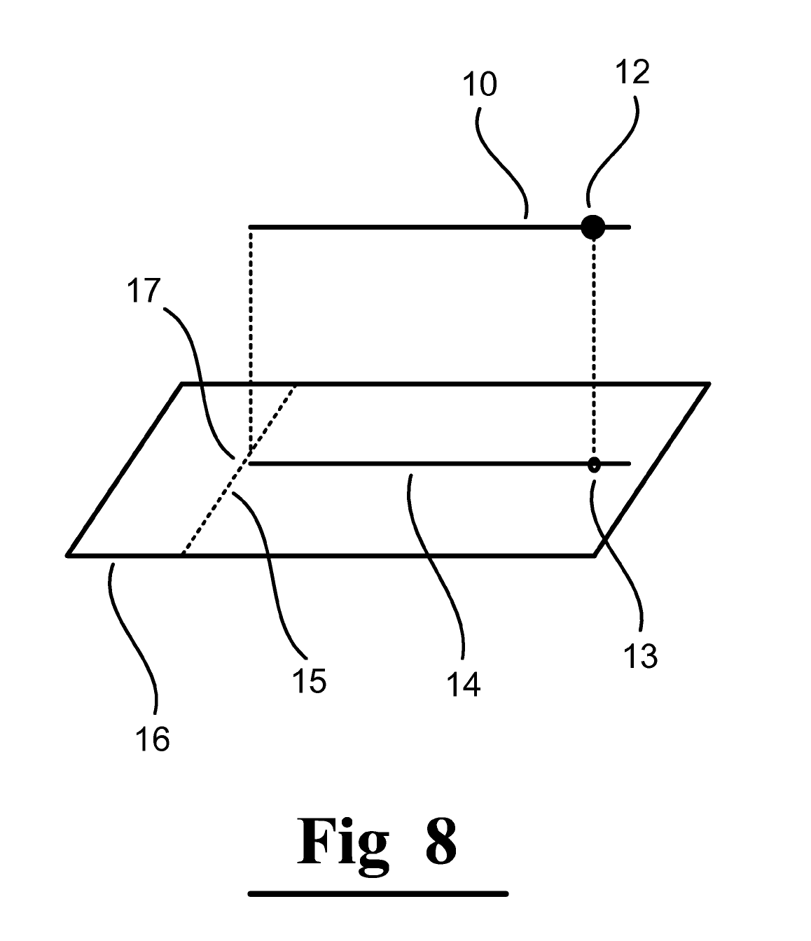

FIG. 6 is a simplified illustration of an appearance of an exemplary bent tool (10) in an exemplary radiographic image, in accordance with some embodiments of the present invention.

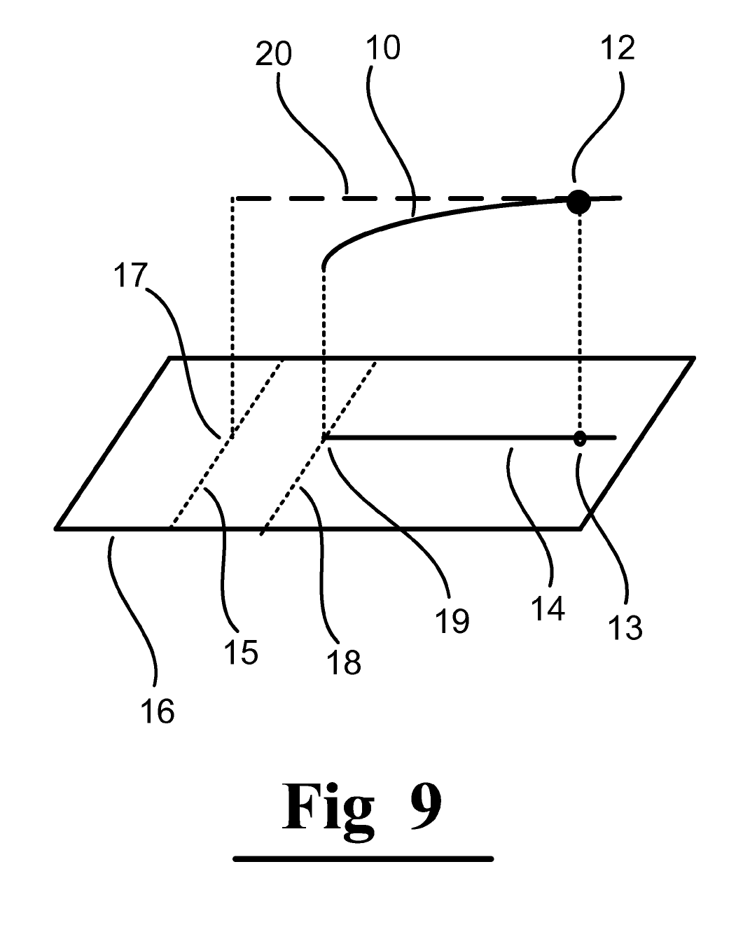

FIG. 7 is an illustration of an expected trajectory (11) of the exemplary tool (10) in FIG. 6, in accordance with some embodiments of the present invention. As illustrated in the Fig, a bent tool may be expected to continue along an arced path. In other scenarios, a bent tool may continue along a straight path after bending. A determination of an expected trajectory of a bent tool may depend on many factors, such as the nature of the tool (e.g. material, shape and construction), the nature of the tissue it is within, etc. As further explained below, according to some embodiments, mathematical models may be used to assist in making the determination.

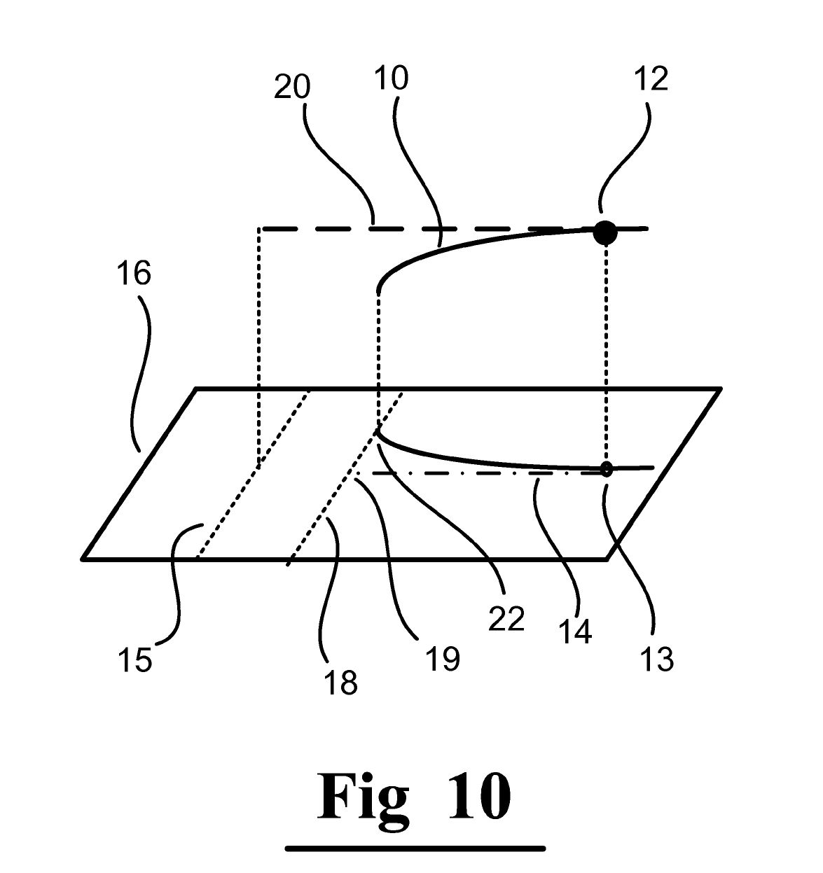

FIG. 8 illustrates an exemplary x-ray image (16), of a tool (10) which is unbent, and the projected image of the tool (14) on the x-ray image. FIG. 8 illustrates a marker (12) on the tool (10) and the projected x-ray image (13) of the marker (12). The projected x-ray image of the tool's tip (17) reaches the dashed line (15).

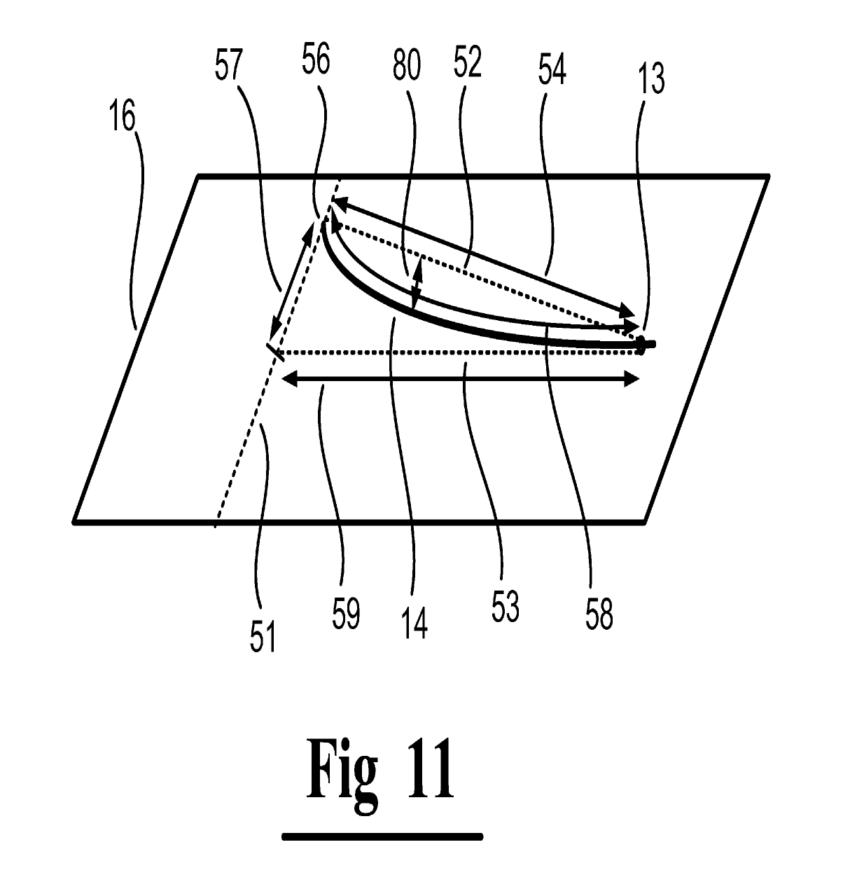

FIG. 9 illustrates an exemplary x-ray image (16) of the same exemplary tool (10) as in FIG. 8, however, in this case the tool is illustrated as bent in a direction perpendicular to the image plane (16). The projected image (14) of the tool (10) is again a straight line, as was in the case of a straight tool shown in FIG. 8. The marker (12) and its projected x-ray image (13) are also located in the same place as in FIG. 8, however, the projected image of the tool's tip (19) reaches the new dashed line (18) and not the dashed line (15) as was when the tool was straight as in FIG. 8 (shown here as a dashed line (20)). The length of the tool's projected image from point (13) (the marker projected x-ray image) to point (19) (the tools tip projected image) is shorter than the length of the tool's projected image from point (13) to point (17) when the tool is straight. Accordingly, the length of the tool's projected image in relation to the expected length if the tool was straight, may be used by the system controller for calculating and estimating the amount of perpendicular bending of the tool.

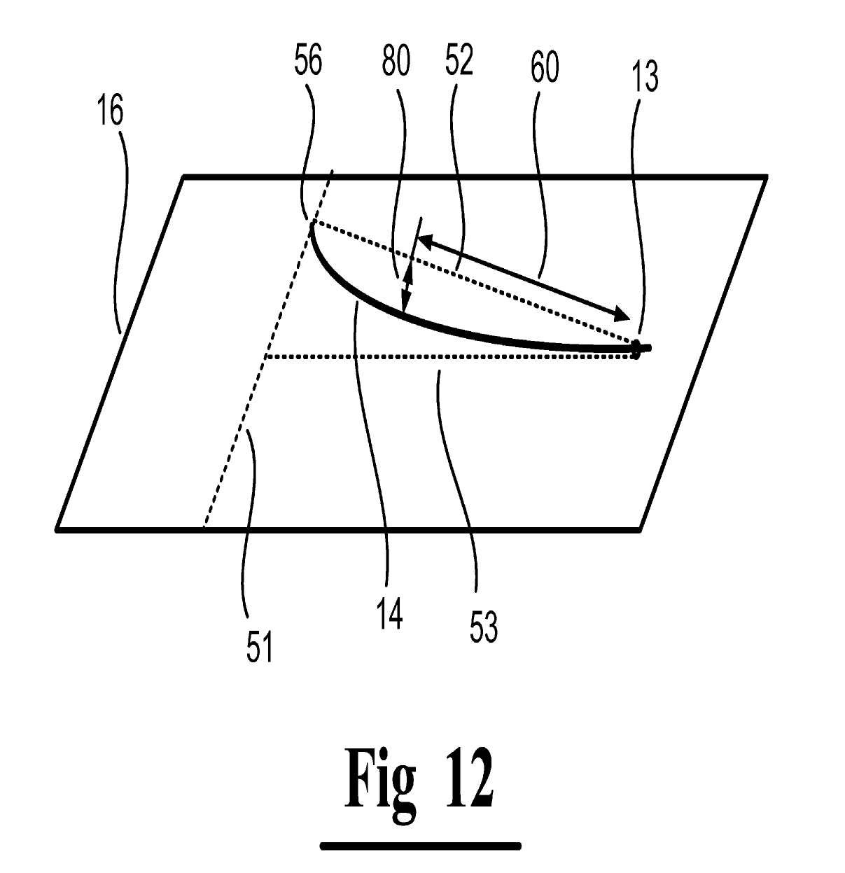

FIG. 10 illustrates an exemplary x-ray image (16) of the same tool (10) as in FIG. 8 and FIG. 9, but in this case the tool is bent in a direction which is both perpendicular and parallel to the image plane (16). The projected image (14) of the tool (10) is a bent line as opposed to a straight line as was in the case of a straight tool in FIG. 8, and as opposed to a straight line as was in the case of a tool bent only in a perpendicular direction to the image plane (16), as shown in FIG. 9. The marker (12) and its projected x-ray image (13) are located in the same place as in FIG. 8 and FIG. 9, but the projected image of the tool's tip reaches dashed line (18) at point (22) and not at point (19) as was the case in a tool bent in a direction perpendicular to the image plane (16) shown in FIG. 9, and not the dashed line (15) as was when the tool was straight as in FIG. 8 (shown here as a dashed line (20)). The distance from point (13) (the projected x-ray image of the marker (12)) to dashed line (18) is equal to the distance from point (13) to dashed line (18) in the case of the tool bent in a perpendicular direction to the image plane (16) shown in FIG. 9, but the point (22) in which the tip of the projected x-ray image of the tool bent both in the perpendicular and parallel direction to the image plane (16) meets dashed line (18) is different than point (19) in which the tip of the projected x-ray image of the tool bent in a perpendicular direction to the image plane (16) meets dashed line (18).

The distance between point (13) (the projected x-ray image of the marker (12)) to dashed line (18) (i.e. the distance between 15 to 18) may be used by the system controller for calculating and estimating the amount of bending of the tool in the perpendicular axis. The location in which point (22) (the projected x-ray image of tool's tip) meets dashed line (18) (i.e. the distance from 22 to 19) may be used by the system controller for calculating and estimating the bending of the tool in the parallel axis, and thus, the orientation in which the tool has bent in the three dimensional coordinate set.

In other words, the appearance of a tool in a radiographic image may be analyzed to determine the bending of the tool, wherein sideways deviations from center may be used to determine bending along a parallel axis and deviations of size in the image may be used to determine bending along a perpendicular axis, thereby, by combining the two, a 3D position and orientation may be obtained.

It should be understood that a tool may not be parallel to the image surface. Clearly, in such cases, calculations described herein may be modified to account for the differences in the 2D measurements (lengths) of the tool within 2D images resulting from the angle between the tool and the image plane. For example, if a tool is angled upward in relation to the image plane, its appearance in a radiographic image may be shorter than it would be if the tool was parallel to the image plane. Such distortions may be calculated using known in the art geometric calculations. For simplicity, within the present description examples of parallel tools are presented. It should be clear that this is done for the purpose of clarity and all such examples should be understood to include the non-parallel cases, in which the necessary modifications to the calculations may be added.

The system controller may be adapted to receive a two dimensional image or model of the tool from the image processor and calculate or otherwise derive a three dimensional shape of the tool using different measurements in the two dimensional image. The system controller may also use physical information of the tool entered from an external input for calculating the three dimensional shape of the tool. The physical information may include for example, physical dimensions of the tool and the tool's shape.

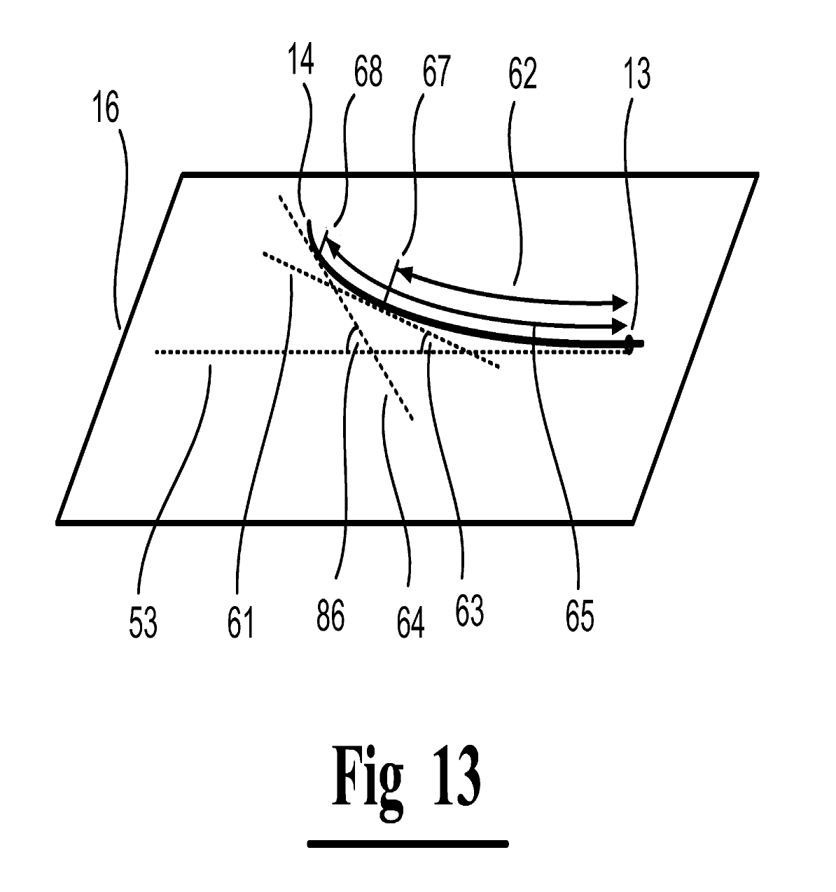

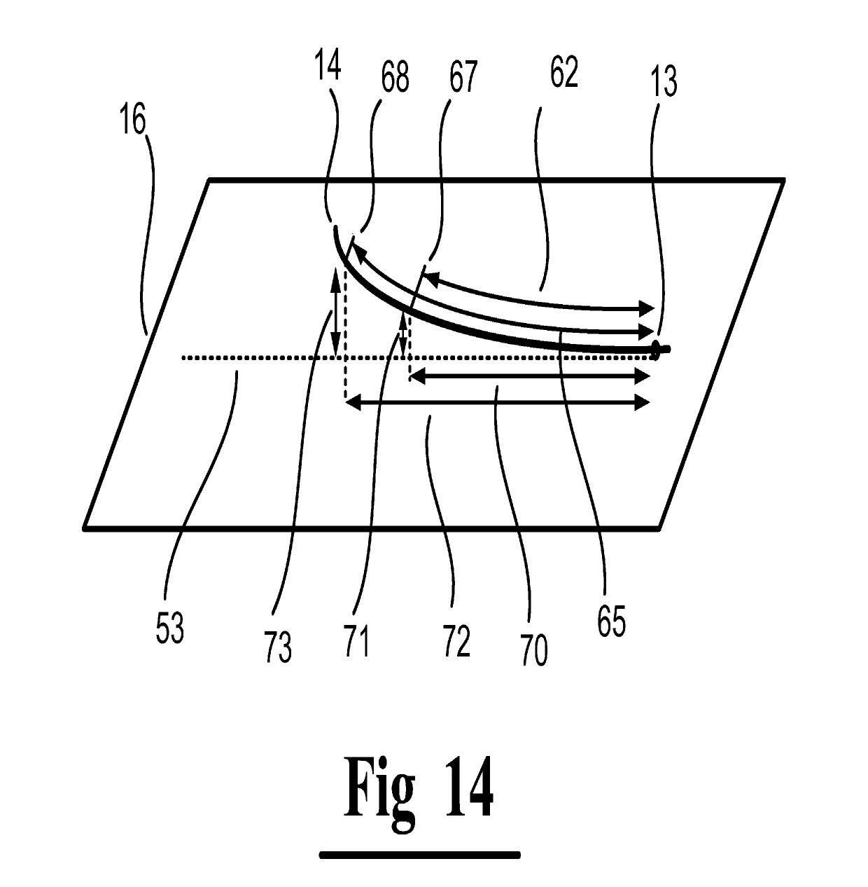

FIGS. 11, 12, 13 & 14 show exemplary two dimensional x-ray images of the tool (14). In the figures examples of certain measurements are shown which may be used by the system controller for calculating the three dimensional shape of the tool. Point (13) is a projected image of a marker at the proximal end of the tool. Dashed line (53) is a virtual line parallel to the projected image of the proximal end of the tool. Dashed line (51) is a virtual line perpendicular to dashed line (53) which crosses the tip of the projected image of the tool (14). Dashed line (52) is a virtual line connecting the projected image of the marker on the tool (13) and the point where the tip of the projected image of the tool (14) touches dashed line (51).

The system controller may extract from the two dimensional image among other measurements and other data extracted from the image:
1) The distance (59) between the projected image of a marker (13) and dashed line (51).
2) The length (58) of the curved line (14) which is a projected image of the tool, between the projected image of the marker (13) and the tip of the projected image of the tool (14).
3) The distance (54) between the projected image of the marker (13) and the tip of the projected image of the tool (14).
4) The distance (57) between the intersection of dashed lines (51) and (53) and the tip of the projected image of the tool (14).
5) The largest distance (80) between the projected image of the tool (14) and the dashed line (52).
6) The distance (60) between the projected image of the marker (13) and the point on dashed line (52) which has the largest distance from the projected image of the tool (14).
7) The distance (62) and (65) between the projected image of a marker (13) and certain points (67) and (68) respectively along the projected image of the tool (14).
8) The angles (63) and (86) between the tangents (61) and (64) respectively, to the projected image of the tool (14); and dashed line (53).
9) The distance (71) and (73) between certain points (67) and (68) respectively along the projected image of the tool (14) and the dashed line (53).
10) Distance (70) and (72) between the projected image of a marker (13) and the lines perpendicular to dashed line (53) that are crossing points (67) and (68) respectively on the projected image of the tool (14).

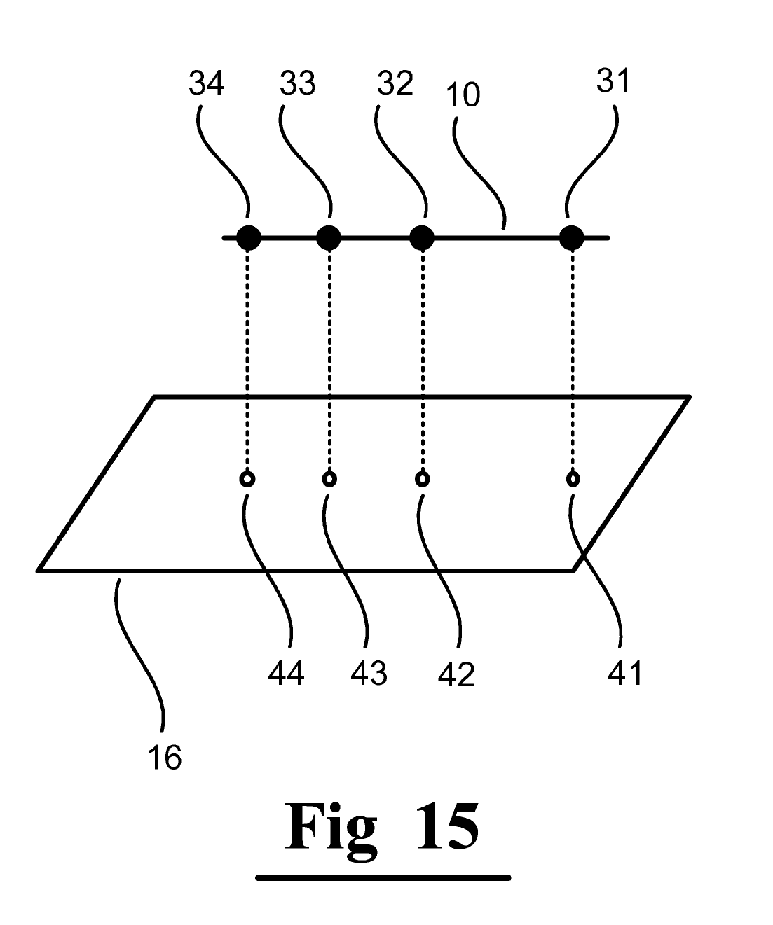

FIG. 15 describes another exemplary embodiment of the present invention. In this embodiment the tool (10) may have certain markers (31), (32), (33) and (34) on certain points on the tool. The markers may be made of a material visible in radiographic images or otherwise have an appearance identifiable in a radiographic image. FIG. 15 illustrates the projected x-ray images (41), (42), (43) and (44) of markers (31), (32), (33) and (34) respectively. By using markers on the tool certain measurements may be made by the system controller between each two x-ray projected marker images. The system controller may also measure distances between each marker and other points on the x-ray image (16) as was explained for instance in FIGS. 11, 12, 13 & 14. By knowing the physical relationship between the markers (inputted to the system controller from an external input) and correlating it with the two dimensional x-ray image, the system controller may determine the shape and orientation of the tool within a three dimensional coordinate set. The system controller may also correlate the two dimensional x-ray image (16) with a two dimensional projection of the three dimensional bent and swiveled/rotated skeleton representation of the tool in order to determine the three dimensional shape and orientation of the tool.

According to further embodiments, the system controller, and/or image processing logic functionally associated therewith may be further adapted to determine and/or predict, or assist in determining and/or predicting, the position and/or orientation of a surgical tool based on mathematical models and formulas describing: (1) the movement of tools within a human anatomy, and (2) the deformation (e.g. bending) of tools within a human anatomy. For example, a mathematical model representing the normal bending of a drill bit when contacting bone at a given angle may be used to predict the upcoming bending of a drill bit as this bit approaches a bone at the given angle. In simple terms, the system may be adapted to predict the movement and deformation of a surgical tool during a medical procedure based on a current radiographic image by first determining the current position, orientation and trajectory of the tool, then determining the tissue with which the tool is expected to come in contact and then using a mathematical model describing the movement and deformation of such a tool when encountering such a tissue to predict the future position, orientation and trajectory of the tool. In this manner, a surgeon may be advised of the expected destination at which the tool will arrive if the surgeon continues along the current path (i.e. pushes forward).

According to further embodiments, such models and formulas may be tool specific (e.g. one model for a drill bit, one for a scalpel, one for a guide wire, etc.) and/or tool material specific (e.g. one model for steel, one for aluminum, one for titanium, etc) and/or may factor the type and/or nature/characteristics of a specific tool in question. Further, models may be designed to factor the deformation of a tool or tool element after bending (some tools may not return to their previous form after bending—e.g. plastic tools). Such models may also be organ/tissue specific and/or may factor the type and nature/characteristics of organs/tissues and the expected effect upon the tool of interacting with the specific organ/tissue. For example, different models (or variations of models) may be provided for different types of bones (e.g. one model for femurs, one for ribs, one for skulls, etc), and/or for different tissues (e.g. one model for bone, one for cartilage, one for muscle tissue, etc). Alternatively, models may include variables dependent upon the tissue/organ in question. Furthermore, such models and formulas may yet further provide for anatomical data relating to the specific patient and/or organ/anatomical-element in contact with the tool. For example, models may be designed to factor patient weight, age, gender, bone mass, etc. Furthermore, such models may factor previous measurements performed in relation to the particular patient and/or tool, possibly in real time. In other words, the system may “learn” in order to improve the accuracy of its predictions.

According to yet further embodiments, the system controller, and/or image processing logic functionally associated therewith may be yet further adapted to extrapolate data relating to the above described mathematical models and formulas from previous tool tracking performed by the system and current tool tracking being performed by the system. For example, based on the movement of a tool when first encountering a harder tissue in a given patient, the expected movement of the tool when encountering the next hard tissue or a model of same may be determined/modified.

Such models and formulas and modifications/updates/profiles for these models may be stored in a functionally associated data storage.

According to some further embodiments, multiple images may be analyzed in conjunction and/or data from one image may be used to assist in analyzing a second image. For example, two images captured from different viewing angles may analyzed by triangulation to determine 3D position of tools and/or anatomical elements, or two images captured at different points in time may be used to assist in determining trajectory/movement of a tool, etc.

While certain features of the invention have been illustrated and described herein, many modifications, substitutions, changes, and equivalents will now occur to those skilled in the art. It is, therefore, to be understood that the appended claims are intended to cover all such modifications and changes as fall within the true spirit of the invention.

I claim:

1. A system for determining the position and orientation of a surgical tool, the system comprising:
a communication module adapted to receive a radiographic image of a surgical tool within or in proximity to a patient;
processing circuitry functionally associated with said communication module and comprising:
first image processing logic adapted to identify appearances of the surgical tool within the radiographic image; and
second image processing logic adapted to extrapolate, based on the identified appearances: (1) a position and orientation of the surgical tool;
and (2) an expected trajectory of the surgical tool.

2. The system according to claim 1, wherein said second image processing logic is adapted to determine and factor deformations of the surgical tool.

3. The system according to claim 1, wherein said second image processing logic is further adapted to extrapolate, based on the identified appearances: (1) a three dimensional (3D) position and orientation of the surgical tool; and (2) an expected 3D trajectory of the surgical tool

4. The system according to claim 3, wherein the surgical tool includes markings visible in a radiographic image and the appearance of the markings in the radiographic image are used by said second image processing logic to determine a (3D) position and orientation of the surgical tool.

5. The system according to claim 1, wherein said second image processing logic further extrapolates an expected future position of the tool.

6. The system according to claim 5, further comprising a rendering module for rendering, upon a display, an image: (1) of the tool, (2) the extrapolated position and orientation of the tool, (3) the expected trajectory of the tool, (4) the extrapolated future position of the tool and (5) anatomical elements of the patient in proximity to the tool.

7. The system according to claim 1, further comprising a data storage of mathematical models describing: (1) the movement of tools within a human anatomy, or (2) the deformation of tools within a human anatomy.

8. The system according to claim 7, wherein said mathematical models factor an effect of an interaction with different types of human tissue upon the movement or form of the tool.

9. The system according to claim 7, wherein said mathematical models are used by said second image processing logic to extrapolate expected future positions of the tool.

10. The system according to claim 7, wherein parameters relating to said mathematical models are updated during a medical procedure.

11. The system according to claim 1, wherein determining a 3D position of the surgical tool includes comparing the appearances of the tool to two dimensional projections of a 3D model of the tool.

12. A method for determining the position and orientation of a surgical tool, the method comprising:
capturing a radiographic image of a surgical tool within or in proximity to a patient;
identifying appearances of the surgical tool within the radiographic image;
automatically extrapolating, by processing circuitry, based on the identified appearances:
(1) a position and orientation of the surgical tool; and
(2) an expected trajectory of the surgical tool.

13. The method according to claim 12, further comprising extrapolating, by processing circuitry, an expected future position of the tool.

14. The method according to claim 13, further comprising rendering, upon a display, an image: (1) of the tool, (2) the extrapolated position and orientation of the tool, (3) the expected trajectory of the tool, (4) the extrapolated future position of the tool and (5) anatomical elements of the patient in proximity to the tool.

15. The method according to claim 13, further comprising using, for extrapolating an expected future position of the tool by the processing circuitry, mathematical models describing: (1) the movement of tools within a human anatomy, or (2) the deformation of tools within a human anatomy.

16. The method according to claim 15, further comprising factoring, within said mathematical models, an effect of an interaction with different types of human tissue upon the movement or form of the tool.

17. The method according to claim 16, wherein extrapolating the expected future position of the tool includes factoring a type of human tissue the tool is expected to encounter.

18. The method according to claim 15, further comprising updating parameters relating to said mathematical models, during a medical procedure.

19. The method according to claim 12, further comprising determining and factoring deformations of the surgical tool.

20. The method according to claim 12, further comprising marking the tool with markings visible in a radiographic image.
